(19) United States
(12) Patent Application Publication
(10) Pub. No.: US 2019/0192011 A1
(43) Pub. Date: Jun. 27, 2019

(54) WEARABLE HEALTH MONITORING DATA SERVICE

(71) Applicant: Playtabase,LLC, Minneapolis, MN (US)

(72) Inventors: David Lee York, Yuba City, CA (US); Peter Eggeman Obringer, San Jose, CA (US); Jose Eduardo Basa, Minneapolis, MN (US); Robert Damien Betz, JR., Jamestown, OH (US); Chris James Rabuse, Lino Lakes, MN (US); Susan Elizabeth Rehl, Burnsville, MN (US); Jake Jones Claypool, Minneapolis, MN (US)

(21) Appl. No.: 16/181,638

(22) Filed: Nov. 6, 2018

Related U.S. Application Data
(60) Provisional application No. 62/581,929, filed on Nov. 6, 2017.

(51) Int. Cl. A61B 5/0205; H04Q 9/00; H04L 29/08; G06F 3/0488; G16H 40/67; A61B 5/00; A61B 5/145; A61B 5/11

(52) U.S. Cl. CPC A61B 5/0205; H04Q 9/00; H04L 67/12; G06F 3/04883; G16H 40/67; A61B 5/681; A61B 5/02438; A61B 5/7435; A61B 5/7475; A61B 5/14542; A61B 5/1112; A61B 5/7221; H04Q 2209/40; A61B 5/0022

(57) ABSTRACT

Systems and techniques for a wearable health monitor device and a wearable health monitoring data service are disclosed here. Sensor data may be periodically collected from a sensor array. The sensor data may be stored in a local database of the wearable health monitor device. The sensor data may be transmitted to a cloud-based platform upon detection of a strong network connection. An evaluation of the sensor data may be received for display on a display device of the wearable health monitor device.

### CLAIM OF PRIORITY

This application claims the benefit of priority to U.S. Provisional Application Ser. No. 62/581,929, filed on Nov. 6, 2017, which is incorporated herein by reference in its entirety.

### TECHNICAL FIELD

Embodiments described herein generally relate to wearable device configuration, data management, and data analysis, in some embodiments, more specifically to a wearable health monitoring data service.

### BACKGROUND

Wearable devices may collect data about a user and their activities and surroundings. A wearable device may be configured in a specific manner to maximize efficiency in collecting data that may be used to evaluate performance of a wearer. The data collected by the wearable may be stored and analyzed to provide the wearer or another individual with information regarding the performance of the wearer.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of a wearable health monitor device and a system for a wearable health monitoring data service, according to an embodiment.

FIGS. 2A, 2B, and 2C illustrate an example of a press and hold user interface for a wearable health monitoring data service, according to an embodiment.

FIG. 3 illustrates an example of a process for data management for a wearable health monitoring data service, according to an embodiment.

FIG. 4 illustrates an example of a process for battery optimization for a wearable health monitoring data service, according to an embodiment.

FIG. 5 is a block diagram illustrating an example of a machine upon which one or more embodiments may be implemented.

### DETAILED DESCRIPTION

As the population ages and technology improves, there may be opportunities that seniors to lead more independent lives. As people age, they may develop or be more susceptible to health issues (e.g., disease, illness, etc. injury (e.g., due to falls, decreased activity levels, etc.). Close monitoring of the activity and vital signs of a senior may allow the senior or their caregiver to identify risks. The senior or their caregiver may then take appropriate steps to promote changes to daily activities that may reduce the risk of developing or worsening illness and injury. Traditionally, continuous monitoring of a senior may be facilitated by residency in a care facility which may have physical and emotional consequences for the senior. The senior may feel confined and the continued monitoring may be viewed as undignified.

The present subject matter provides a wearable health monitor device and corresponding data service that may be wont by a senior that may monitor the activity and vital signs of the senior. Data may be collected regarding the senior without the need for confinement in a specialized facility or conventional vital sign collection by a caregiver. Thus, the senior may be able to continue living at home or in a less confining facility such as a private unit in an assisted living facility. The data for the senior may be collected and analyzed and compared against statistical models to identify if the activity and vital signs of the senior are meeting the needs of a current care plan and/or whether the data indicates the senior is at an increased risk of health degradation or injury.

Sensor data may be collected from the wearable health monitor device for the purpose of improving an individual's health and the healthcare services they're offered. As an example, consider a senior individual over the age of 65 years old. Older users may have mobility challenges, mental challenges, or may not be familiar with current technology. Thus, user interfaces may be designed to provide streamlined interaction and easy to understand output. Thus, the present solution may provide value to the senior by providing an easy to use user interface and readily consumable output. In addition, an easy to use interface for activating an emergency response protocol may be provided via the wearable allowing rapid access to a personal emergency response service.

The present subject matter may be implemented as a software architecture platform in the wearable health monitor device which may allow for flexibility in developing services and applications to run on the wearable health monitor device. The platform may include a data service that may capture and transmit sensor data to a cloud computing platform (e.g., an internet connected cluster of computing devices, etc.). The platform may include a set of battery optimization algorithms allowing for increased device efficiency and increased run time between charging.

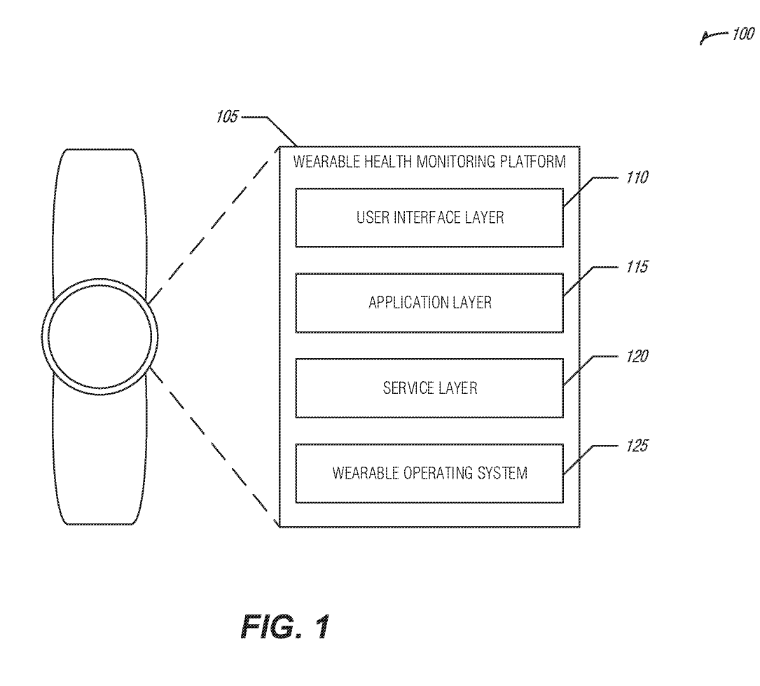

FIG. 1 illustrates an example of a wearable health monitor device 100 and a system 105 for a wearable health monitoring data service, according to an embodiment. The wearable health monitor device 100 may be a smartwatch, wristband, smart clothing, or other device including sensors capable of capturing user activity data and vital sign data (e.g., gyroscope, accelerometer, magnetometer, infrared sensor, camera, microphone, gas sensor, photodetector, etc.).

The system 105 may be implemented in the wearable health monitor device 100. The system 100 may be a wearable architecture such as a wearable health monitoring platform that may include a user interface layer 110, an application layer 115, a service layer 120, and a wearable operating system 125. The system 105 may include a set of machine instructions stored in machine readable memory of the wearable health monitor device 100 that, when executed by processing circuitry of the wearable health monitor device 100, perform a variety of operations including the capture and transmission of sensor data.

The system 105 may be configured to allow for flexibility of the type of data captured and the manner of data transmission. In an example, data capture may include resident sensors on the wearable health monitor device and may include other devices providing sensor readings or data packets that the wearable health monitor device 100 may be able to capture. The other devices including sensors may be connected to the wearable health monitor device 100 using a variety of connectivity techniques such as, for example, Bluetooth, NFC, wireless, cellular, etc. Data transmission may utilize a cloud environment as an end point or any other connected data storage and analysis environment.

The system 105 may include a variety of layers. The individual layers may provide a separation of processing operations that may allow for asynchronous development of components within each layer. Such separation may be provided using interface points between components or services of each layer. Each component or service may include a set of instructions that may be a part of (or may work in conjunction with) processing circuitry that may cause hardware and software components in the wearable health monitor device 100 to perform a variety of operations.

The wearable operating system 125 may provide basic operating instructions that provide basic input, basic output, post, and other basic operability functions for the wearable health monitor device 100. The wearable device 100 may include communication hardware such as, for example, a cellular network transceiver. The wearable operating system 125 may facilitate communication between components of the system 105 and the communication hardware.

The service layer 120 may work in conjunction with the wearable operating system 125 to manage data capture and data transmission functions of the wearable heath monitor device 100. For example, the service layer 120 may manage an array of sensors included in the wearable health monitor device 100 to collect sensor readings at various intervals and transmit the sensor data from the wearable health monitor device 100 to the cloud-based service.

In an example, the service layer 120 may include a capture data service that first captures sensor data from the wearable health monitor device. This would include data such as battery level, heart rate readings, step count reading and GPS location readings. This capture data service would capture the information and manage the local storage of the data.

In an example, the service layer 120 may include a transmission data service that would track available connections (e.g., cellular, etc.) and transmit data when the connection allows.

The service layer 120 may include a scheduling data service that coordinates the execution of the capture data service and once completed, initiates the data transmission service.

The application layer 115 may provide interfaces between the wearable operating system 125, the service layer 120, the user interface layer 110, the cloud-based service, and other devices. In an example, the application layer 115 may include a wireless communication application (e.g., Bluetooth, etc.) that connects to a wireless enabled device (e.g., a weight scale, a blood pressure monitor, etc.) to collect sensor readings from sensors included in the devices. The wireless communication application may utilize the capture data service of the service layer 120 to store the sensor readings in a local storage medium.

The user interface layer may provide instructions for generating graphical interfaces with which the user may interact with the wearable health monitor device 100. In an example, the user interface layer 110 may include a wearable user interface that allows the user to initiate the heart rate sensor and to place an emergency call using a Press and Hold User Interface.

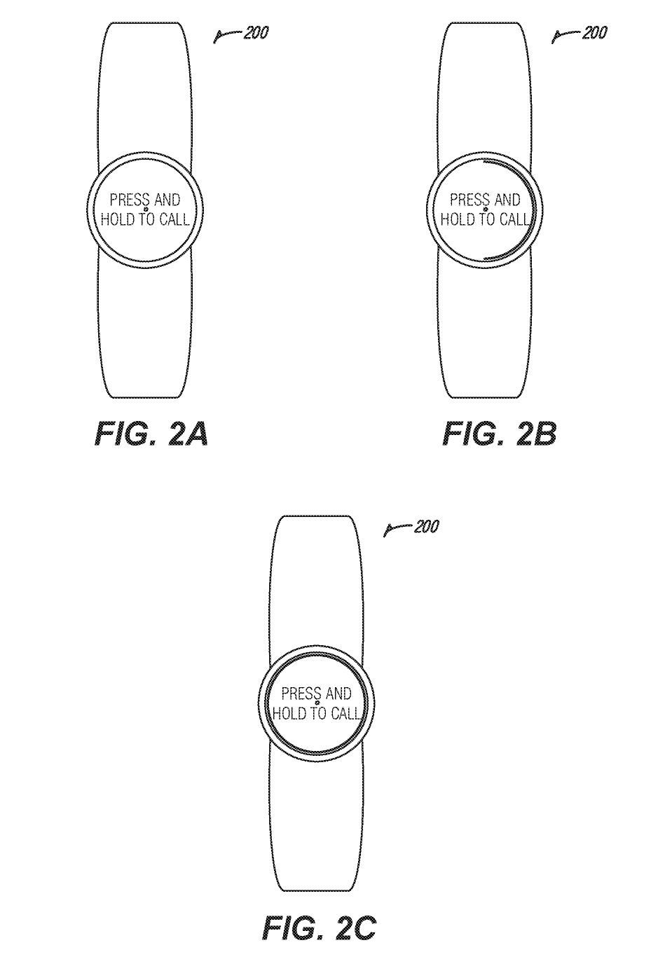

FIGS. 2A, 2B, and 2C illustrate an example of a press and hold user interface 200 for a wearable health monitoring data service, according to an embodiment. A user may touch the screen (or otherwise activate an emergency call feature) of a wireless health monitor device. The press and hold user interface 200 may be displayed (e.g., as shown in FIG. 2A). As the user holds the screen, the press and hold user interface 200 may provide a graphical user interface element showing the progress of the request (e.g., as shown in FIG. 2B). The delay in activating the call may prevent false activations for users that may have mobility issues or may be prone to accidental screen touches (e.g., via bumping, accidental swipes, etc.). The delay and corresponding visual elements may be adjusted for individual users and for groups of users depending on conditions and needs of the user and group. For example, a delay may be shortened for a user with a heart condition while the delay may be increased for a user with hand tremors, and so forth. Once the delay has been reached, the visual indicator may reach a final stage (e.g., as shown in FIG. 2C) and the call may be placed.

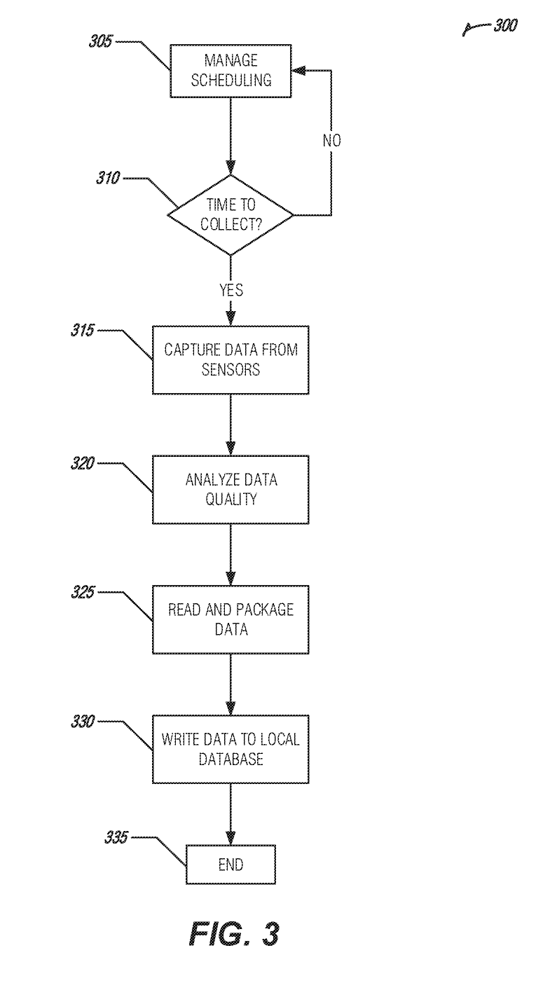

FIG. 3 illustrates an example of a process 300 for data management for a wearable health monitoring data service, according to an embodiment.

The present subject matter may include a wearable data service. The wearable data service may include, a set of functions and algorithms that manage data capture of a wearable health monitor device's sensors, a set of functions and algorithms that analyze the quality of the sensor reading, a light weight local relational database that provides persistent storage of the sensor readings and allows for ingress and egress of data as well as flexibility to add or modify sensor readings, a set of functions and algorithms that manage the reading and packaging of sensor readings from the local relational database, a set of functions and algorithms that manage the transmission of data packets from the wearable to non-local cloud storage via cellular, Wi-Fi etc.

The wearable data service may include a set of functions and algorithms that manage the schedule for data management functions such as, for example, initiating sensor readings, analyzing sensor readings, interfacing with the local relational database, reading and packaging of local data, transmitting local data packets to non-local cloud storage, etc. The wearable data service may optimize a wearable health monitor device to perform operations such as, for example, collection of heart rate reading(s) from the wearable health monitor device, storage of the heart rate reading(s) locally on a storage medium of the wearable health monitor device, and transmission of the heart rate reading(s) from the wearable health monitor device to non-local cloud storage via a network (e.g., wireless network, near-field communication, wired network, short-wave radio, etc.).

The wearable data service may utilize a schedule function to periodically check the local time and invoke a heart rate check every 15 minutes. The service then invokes a function that listens for the latest heart rate reading (e.g., at operation 305). Upon receiving the latest heart rate reading, the service checks against the current local time. If the interval is greater than 15 minutes (e.g., as determined at decision 310), it invokes a heart rate collection function (e.g., at operation 315). The heart rate collection invokes a function that attempts to collect a heart rate for a period of 10 seconds. If successful, it records the reading and proceeds. If unsuccessful, it retries the same function but adjusts the period to 15 seconds (e.g., at operation 305). This retry loop can be adjusted dynamically. The retry loop also has an unsuccessful threshold level that when reached, will record an unsuccessful attempt.

Upon either a successful attempt or unsuccessful attempt, the service then invokes a data quality check that evaluates the accuracy of the reading (e.g., at operation 320). For any successful reading, it uses a custom algorithm to label the reading as “accurate” or “not accurate” (e.g., at operation 325). For any unsuccessful reading, it labels the reading as “not accurate”. After labeling each reading, the service compiles a SQL statement to insert the record into a local persistent database (e.g., at operation 330). The service invokes the statement. The service writes to a local audit log to determine if the statement was successful and the process 300 ends (e.g., at end 335).

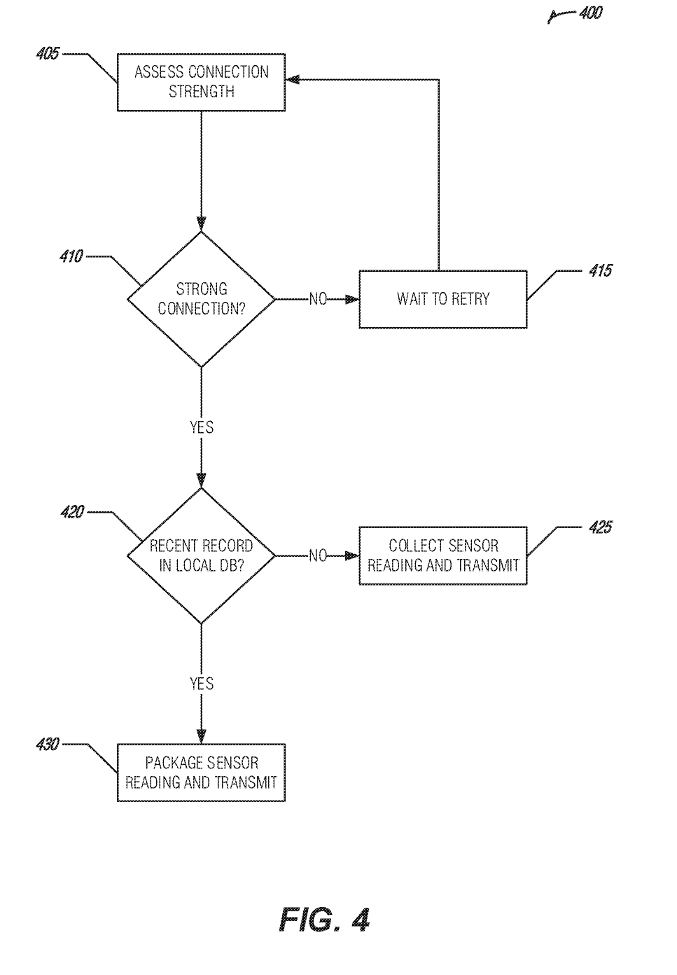

FIG. 4 illustrates an example of a process 400 for battery optimization for a wearable health monitoring data service, according to an embodiment. A variety of battery optimization techniques may be used to maximize the run time of the wearable health monitor device between recharge. These techniques may increase the sensor data collection cycles that may be performed between charges and may reduce the frequency with which a senior or caregiver must recharge the wearable health monitor. This may increase the ease of use and independence of seniors using the devices, especially those with arthritis or other ailments affecting dexterity, by reducing potentially unwanted visits into their personal space (e.g., private apartment, etc.

The battery optimization algorithms are based on the operating characteristics of the wearable health monitor device. Power draw of individual sensors (e.g., heart rate sensor, GPS, etc.) of the wearable health monitor device may be approximated. Power draw of individual functions (e.g., data transmission request, etc.) may be approximated. Characteristics of individual functions (e.g., accuracy of sensor readings, connection strength, etc. may be assessed. The power draw approximations and characteristics may be used to model and test different combinations of sensor readings and function calls to assess performance relating to approximate power draw, reliability, and accuracy of a modeled combination. Based on characteristics of individual sensor/functions and combination(s), new data collection processes may be developed based on the modeling and testing to collect data while incurring lowest possible power draw.

In an example, connection strength may be used in optimizing battery usage in data transmission. This may result in shorter data transmission times resulting in lower battery usage. The connection strength may be assessed (e.g., at operation 405). Connection strength may be based on signal strength, available bandwidth, or other characteristics of the connection indicative of reduced data transmission times. If the connection is determined not to be strong (e.g., at decision 410), the wearable health monitor device may wait to reattempt data collection (e.g., at operation 315). A threshold may be employed to determine if a connection is strong. For example, if currently detected bandwidth is below fifty percent of an expected available bandwidth value the connect may not be determined as strong while current available bandwidth above fifty percent of the expected bandwidth value may be determined to be a strong connection. If the connection is strong (e.g., as determined at decision 410), it may be determined in a recent record for the sensor collection item in a local database (e.g., at decision 420). If there is not a recent record in the local database, the sensor reading may be collected and transmitted (e.g., at operation 425). If there is a recent record in the local database, the sensor reading may be package and transmitted (e.g., at operation 430).

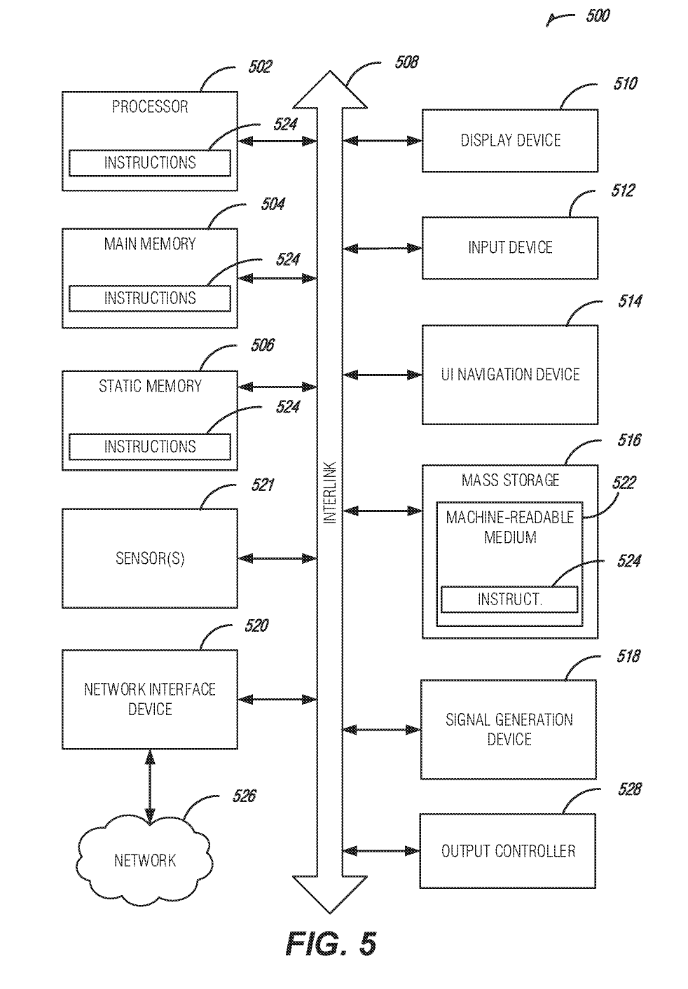

FIG. 5 illustrates a block diagram of an example machine 500 upon which any one or more of the techniques (e.g., methodologies) discussed herein may perform. In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to can out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 500 may include a hardware processor 502 (e.g., a central processing unit (CPU), a graphics processing unit (GPU), a hardware processor core, or any combination thereof), a main memory 504 and a static memory 506, some or all of which may communicate with each other via an interlink (e.g., bus) 508. The machine 500 may further include a display unit 510, an alphanumeric input device 512 (e.g., a keyboard), and a user interface (UI) navigation device 514 (e.g., a mouse). In an example, the display unit 510, input device 512 and UI navigation device 514 may be a touch screen display. The machine 500 may additionally include a storage device (e.g., drive unit) 516, a signal generation device 518 (e.g., a speaker), a network interface device 520, and one or more sensors 521, such as a global positioning system (GPS) sensor, compass, accelerometer, or other sensor. The machine 500 may include an output controller 528, such as a serial (e.g., universal serial bus (USB), parallel, or other wired or wireless (e.g., infrared (IR), near field communication (NFC), cellular, etc.) connection to communicate or control one or more peripheral devices (e.g., a printer, card reader, etc.).

The storage device 516 may include a machine readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 516 may constitute machine readable media.

While the machine readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, Internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

1. A wearable health monitor device for a wearable health monitoring data service, the wearable health monitor device comprising:
at least one processor; and
memory including instructions that, when executed by the at least one processor, cause the at least one processor to perform operations to:
periodically collect sensor data from a sensor array;
store the sensor data in a local database of the wearable health monitor device;
transmit, upon detection of a strong network connection, the sensor data to a cloud-based platform; and
receive, an evaluation of the sensor data for display on a display device of the wearable health monitor device.

2. The wearable health monitor device of claim 1 further comprising instructions to:
generate, upon detection of a touch of the display device of the wearable health monitor device, a push and hold user interface for display on the display device of the wearable health monitor device;
display the push and hold user interface on the display device of the wearable health monitor device;
display, in the push and hold user interface, a graphical user interface element that varies over time the longer a touch of the display device is detected; and
transmit, instructions to initiate a communication session, via a wireless network, based on a lapse of time of the detected touch of the display device being outside a threshold.

3. The wearable health monitor device of claim 1, wherein the strong network connection is a connection to a cellular data network.

4. The wearable health monitor device of claim 1, wherein the sensor array includes at least one sensor of a group of sensors comprising: a heart rate monitor, an oxygen sensor, a global positioning system receiver, a gyroscope, and an accelerometer.

5. The wearable health monitor device of claim 1, wherein the wearable health monitor device is a smartwatch.

6. The wearable health monitor device of claim 1, wherein the evaluation of the sensor data includes determining a quality level of the data and the evaluation of the sensor data is displayed upon a determination that the quality level is outside a threshold.

7. The wearable health monitor device of claim 6, wherein determining the quality level includes evaluating the sensor data against another set of sensor data collected from the sensor array during a retry loop, and wherein the evaluation of the sensor data is displayed upon a determination that the evaluation of the sensor data and the other sensor data indicates the sensor data is stable.

8. At least one non-transitory machine-readable medium including instructions for interaction between a wearable health monitoring device and a wearable health monitoring data service that, when executed by the at least one processor, cause the at least one processor to perform operations to:
periodically collect sensor data from a sensor array;
store the sensor data in a local database of the at least one non-transitory machine-readable medium;
transmit, upon detection of a strong network connection, the sensor data to a cloud-based platform; and
receive, an evaluation of the sensor data for display on a display device of the at least one non-transitory machine-readable medium.

9. The at least one non-transitory machine-readable medium of claim 8, further comprising instructions to:
generate, upon detection of a touch of the display device of the at least one non-transitory machine-readable medium, a push and hold user interface for display on the display device of the at least one non-transitory machine-readable medium;
display the push and hold user interface on the display device of the at least one non-transitory machine-readable medium;
display, in the push and hold user interface, a graphical user interface element that varies over time the longer a touch of the display device is detected; and
transmit, instructions to initiate a communication session, via a wireless network, based on a lapse of time of the detected touch of the display device being outside a threshold.

10. The at least one non-transitory machine-readable medium of claim 8, wherein the strong network connection is a connection to a cellular data network.

11. The at least one non-transitory machine-readable medium of claim 8, wherein the sensor array includes at least one sensor of a group of sensors comprising: a heart rate monitor, an oxygen sensor, a global positioning system receiver, a gyroscope, and an accelerometer.

12. The at least one non-transitory machine-readable medium of claim 8, wherein the wearable health monitor device is a smartwatch.

13. The at least one non-transitory machine-readable medium of claim 8, wherein the evaluation of the sensor data includes determining a quality level of the data and the evaluation of the sensor data is displayed upon a determination that the quality level is outside a threshold.

14. The at least one non-transitory machine-readable medium of claim 13, wherein determining the quality level includes evaluating the sensor data against another set of sensor data collected from the sensor array during a retry loop, and wherein the evaluation of the sensor data is displayed upon a determination that the evaluation of the sensor data and the other sensor data indicates the sensor data is stable.

15. A method for interaction between a wearable health monitor device and a wearable health monitoring data service, the method comprising:
periodically collecting sensor data from a sensor array;
storing the sensor data in a local database of the method;
transmitting, upon detection of a strong network connection, the sensor data to a cloud-based platform; and
receiving, an evaluation of the sensor data for display on a display device of the method.

16. The method of claim 15, further comprising:
generating, upon detection of a touch of the display device of the method, a push and hold user interface for display on the display device of the method;
displaying the push and hold user interface on the display device of the method;
displaying, in the push and hold user interface, a graphical user interface element that varies over time the longer a touch of the display device is detected; and
transmitting, instructions to initiate a communication session, via a wireless network, based on a lapse of time of the detected touch of the display device being outside a threshold.

17. The method of claim 15, wherein the strong network connection is a connection to a cellular data network.

18. The method of claim 15, wherein the sensor array includes at least one sensor of a group of sensors comprising: a heart rate monitor, an oxygen sensor, a global positioning system receiver, a gyroscope, and an accelerometer.

19. The method of claim 15, wherein the wearable health monitor device is a smartwatch.

20. The method of claim 15, wherein the evaluation of the sensor data includes determining a quality level of the data and the evaluation of the sensor data is displayed upon a determination that the quality level is outside a threshold.
